(19) United States
(12) Patent Application Publication
(10) Pub. No.: US 2011/0039319 A1
(43) Pub. Date: Feb. 17, 2011

(54) ENZYME RECYCLE FROM HYDROLYSIS OF LIGNOCELLULOSIC MATERIAL

(76) Inventors: Theodora Retsina, Atlanta, GA (US); Vesa Pylkkanen, Atlanta, GA (US)

Correspondence Address: American Process Inc;Steve Rutherford, 750 Piedmont Avenue NE, Atlanta, GA, 30308, US

(21) Appl. No.: 12/854,869

(22) Filed: Aug. 11, 2010

Related U.S. Application Data
(60) Provisional application No. 61/233,324, filed on Aug. 12, 2009.

(51) Int. Cl. C12P 7/14

(52) U.S. Cl. 435/162

(57) ABSTRACT

A method to reduce enzyme usage for the break down of lignocellulosic material by enzymatic hydrolysis. Enzyme activity is retained and enzymes are recycled back for the hydrolysis of new lignocellulosic material after removal of fermentation products using low temperature distillation.

### CONTINUATION

This is a continuation of provisional patent application No. 61/233,324

### DESCRIPTION

### Field of the Invention

This invention relates, in general, to the enzymatic hydrolysis of lignocellulosic material, and more specifically to the preservation of the enzymes for recycle and reuse after removal of fermentation products.

### BRIEF DESCRIPTION OF THE DRAWINGS

A more complete understanding of the present invention may be obtained by reference to the following detailed description, when read in conjunction with the accompanying drawing wherein:

FIG. 1. illustrates an example of enzyme recycle process configuration. Step sequences may vary for specific applications. Continuous or simultaneous processing is possible.

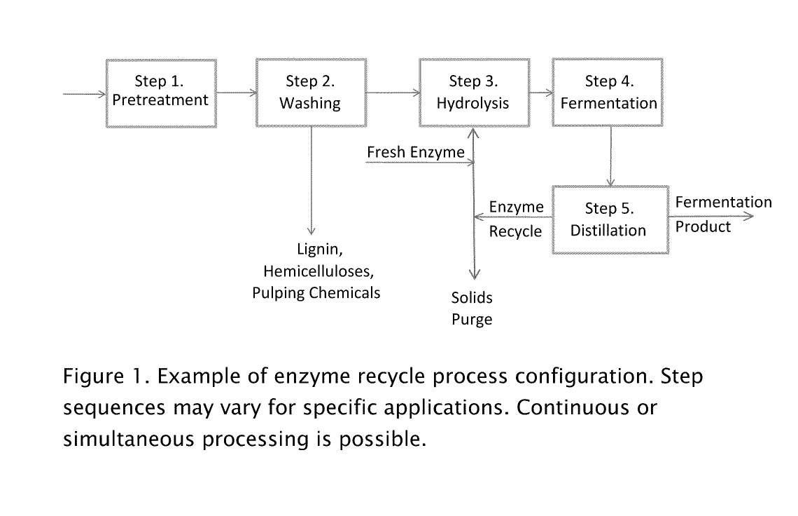

### BACKGROUND OF THE INVENTION

role of lignin in the enzymatic hydrolysis of lignocelluloses was studied by Palonen et al. (J Biotechnol. 2004 January 8; 107(1):65-72), who concluded that the type of pretreatment has a strong impact on non-specific binding of cellulases, cellulose degrading enzymes, to lignocellulose. These enzymes are difficult to recover and recycle from the residue after hydrolysis.

The current inventors propose a method, whereby enzymes are recovered after a fermentation product removal stage, recycled back and reused in the hydrolysis of new lignocellulosic material.

### SUMMARY OF THE INVENTION

Lignocellulosic material is pretreated to lower its lignin content by a pulping process. The pulped lignocellulosic material is washed to separate solid cellulose from the dissolved wood components, lignin and hemicelluloses, and any pulping chemicals.

The cellulose is enzymatically hydrolyzed to fermentable sugars. Sugars are separately or concurrently fermented with an appropriate micro-organism, preferably yeast. Yeast can be separated from the beer after the fermentation.

Beer is distilled to separate and remove the fermentation product, such as ethanol or other biochemical, from the top of the column under a vacuum to prevent the cellulase enzymes from permanently losing their activity.

The cellulases in the bottoms of the distillation column are recycled back to the hydrolysis step. Fresh enzymes may be added to the hydrolysis.

### DETAILED DESCRIPTION OF THE INVENTION

In the first step, pretreatment, lignocellulosic material is treated to lower its lignin content.

For softwood or hardwood this pretreatment is a pulping process reducing the lignin content of the pulped lignocellulosic material preferably to less than 2%. The pretreatment preferably removes also the hemicelluloses to increase the cellulose fraction. The pulping process may be an alkaline or acidic, preferably acid sulfite and most preferably solvent sulfite, where the hemicelluloses can be recovered and processed separately to fermentation products.

The second step, washing, is performed on the pulped lignocellulosic material to separate solid cellulose from the dissolved wood components, lignin and hemicelluloses, and pulping chemicals. The washed cellulose is dewatered for the next step.

In the third step, hydrolysis, the washed cellulose is diluted with an aqueous enzyme cocktail, which includes cellulases, to hydrolyze the cellulose to fermentable sugars. The hydrolysis of cellulose is performed separately or concurrently with the fermentation step, to complete the degradation of cellulose. The solid residues, containing lignin and adsorbed enzymes, are removed or left in the solution. Additives or detergents may be used to release or block the non-specific binding of cellulases to the lignin.

The fourth step, fermentation, is brought to completion on the fermentable sugars using an appropriate naturally occurring or engineered micro-organism, preferably saccharomyses cerevisiae yeast. The yeast is separated from the beer after the fermentation.

The fifth step, distillation, is performed at vacuum to prevent the cellulase from permanently losing their activity while removing fermentation products, preferably ethanol or other biochemical. Cellulase activity decreases at elevated temperature, but can be restored at lower temperatures as long as the cellulase upper temperature limit has not been exceeded. As an example, activity for one type of cellulase occurs between 30 and 70° C., with the peak around 50° C.; therefore the distillation equipment is designed to operate at an absolute pressure below the corresponding cellulase upper temperature limit, i.e., in this example below 31.2 kPa corresponding to 70° C. Distillation at temperatures above the cellulase upper temperature limit results in permanent loss of cellulase activity, as would occur with atmospheric or pressurized distillation.

The bottoms of the distillation column are recycled back to the hydrolysis step. Fresh enzymes are also added to the hydrolysis step to replenish any loss caused by solids purge or decreased enzyme activity. The ability to recycle cellulase results in a scheme that allows high effective enzyme usage with lower overall enzyme application

Steps 3 and 4 as well as steps 3, 4 and 5 may be conducted simultaneously in a continuous step.

Although other modifications and changes may be suggested by those skilled in the art, it is the intention of the inventors to embody within the patent warranted hereon all changes and modifications as reasonably and properly come within the scope of their contribution to the art.

What is claimed:

1. A method for enzymatically hydrolyzing washed cellulose from pulped lignocellulosic material to fermentable sugars, fermenting the sugars, separating fermentation products in a distillation column and recycling the enzymes to hydrolyze new cellulose material.

2. A method according to claim 1, wherein said pulped lignocellulosic material is obtained from an alkaline pulping process.

3. A method according to claim 1, wherein said pulped lignocellulosic material is obtained from an acid pulping process.

4. A method according to claim 1, wherein said pulped lignocellulosic material is obtained from solvent pulping process.

5. A method according to claim 1, wherein said pulped lignocellulosic material is washed to reduce pulping chemical, lignin and hemicellulose content.

6. A method according to claim 1, wherein said fermentable sugars are fermented to ethanol.

7. A method according to claim 1, wherein said fermented ethanol is distilled under vacuum.

8. A method according to claim 1, wherein said fermentable sugars are fermented to a biochemical.

9. A method according to claim 1, wherein said fermented biochemical is distilled under vacuum.

10. A method according to claim 7, wherein said vacuum creates column temperature below the upper limit for permanent enzyme activity loss.

11. A method according to claim 1, wherein said enzymes consists of cellulases.

12. A method according to claim 1, wherein said enzymes are stable at elevated temperatures.

13. A process for pulping and washing lignocellulosic material to a low lignin content, enzymatically hydrolyzing and fermenting the remaining cellulose, and distilling fermentation products under vacuum to retain enzymatic activity in the distillation bottoms for the recycle and reuse of the enzymes on new cellulose material.
